(19) United States
(12) Patent Application Publication
(10) Pub. No.: US 2024/0173016 A1
(43) Pub. Date: May 30, 2024

(54) ASSESSMENT OF TISSUE ABLATION USING INTRACARDIAC ULTRASOUND CATHETER

(71) Applicant: BIOSENSE WEBSTER (ISRAEL) LTD., Yokneam (IL)

(72) Inventors: Roy Urman, Karkur (IL); Zvi Dekel, Zichron Yaakov (IL)

(21) Appl. No.: 18/070,770

(22) Filed: Nov. 29, 2022

(51) Int. Cl. A61B 8/08; A61B 8/00; A61B 8/12; A61B 18/14

(52) U.S. Cl. CPC A61B 8/5246; A61B 8/0841; A61B 8/12; A61B 8/4494; A61B 8/463; A61B 8/483; A61B 18/1492; A61B 2018/00357

(57) ABSTRACT

A system includes a processor and a display. The processor is configured to: receive a first plurality of ultrasound (US) images acquired at a site of an organ from a first plurality of positions and orientations, before performing a medical procedure at the site; receive a second plurality of US images acquired at the site, from a second plurality of positions and orientations, after performing the medical procedure at the site; and identify among the first and second plurality of US images, one or more pairs of first and second US images, respectively, which are acquired from matched orientations, and select a given pair among the pairs, in which a difference between the first and second US images is largest among the identified pairs. The display is configured to display the given pair of the first and second US images to a user.

### FIELD OF THE DISCLOSURE

The present disclosure relates generally to medical devices, and particularly to methods and systems for improving the assessment of lesion formed in tissue ablation using an intracardiac ultrasound catheter.

### BACKGROUND OF THE DISCLOSURE

Tissue ablation is used for treating arrhythmia by applying ablation energy to tissue so as to transform the tissue to a lesion, and thereby, blocking propagation of electrophysiologic waves therethrough. Various techniques have been developed for assessing the quality of lesions formed in ablation procedures.

The present disclosure will be more fully understood from the following detailed description of the examples thereof, taken together with the drawings in which:

### BRIEF DESCRIPTION OF THE DRAWINGS

FIG. 1 is a schematic, pictorial illustration of a catheter-based ultrasound imaging and tissue ablation system, in accordance with an example of the present disclosure;

FIGS. 2A, 2B, 3A and 3B are schematic, pictorial illustrations of intracardiac ultrasound signals applied to heart tissue for assessing the quality of a lesion formed by tissue ablation, in accordance with examples of the present disclosure;

FIG. 4 is a schematic, pictorial illustration of ultrasound images acquired before and after the tissue ablation and displayed to a user, in accordance with an example of the present disclosure; and

FIG. 5 is a flow chart that schematically illustrates a method for assessment of lesion formed by tissue ablation, in accordance with an example of the present disclosure.

### DETAILED DESCRIPTION OF EXAMPLES

### Overview

Arrhythmias in a patient heart may be caused by undesired propagation of an electrophysiological (EP) wave at specific location(s) on the surface of heart tissue. Tissue ablation is used, inter alia, for treating various types of arrhythmia by transforming a living tissue (that enables the propagation of the EP wave) to a lesion that blocks the propagation of the EP wave. The quality and location of the lesion are important for obtaining a successful ablation procedure.

Examples of the present disclosure that are described below, provide techniques for assessing the quality of a lesion formed in an organ, such as in a heart of a patient.

In some examples, a catheter-based ultrasound imaging and tissue ablation system comprises a catheter, a processor and a display device, also referred to herein as a display, for brevity.

In some examples, the catheter comprises a four-dimensional (4D) ultrasound catheter with a distal tip having ultrasound transducers, which are configured to apply US waves to an ablation site on heart tissue. The distal tip is configured to produce, based on US waves returned (e.g., reflected) from the tissue in question, one or more US signals indicative of the shape and morphology of the tissue in question.

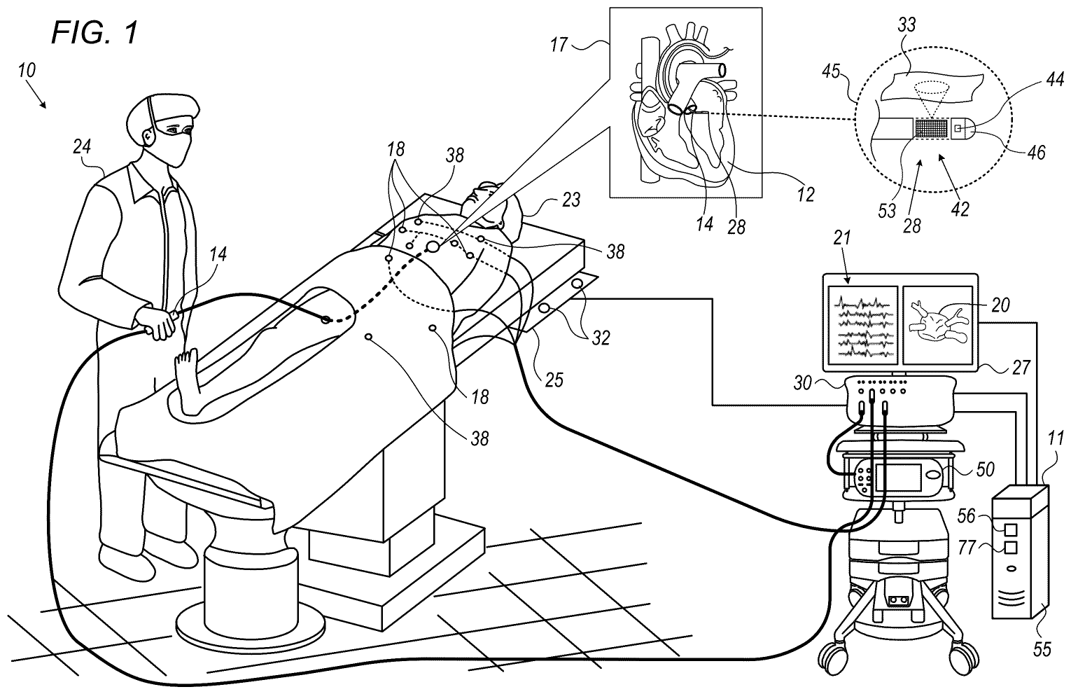

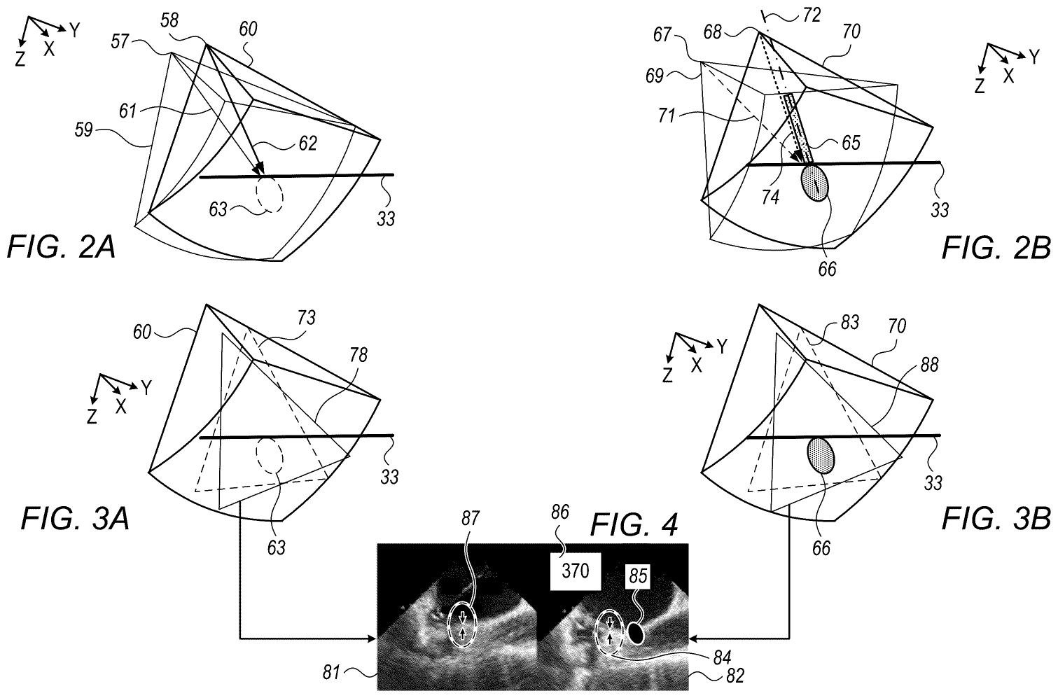

In some examples, the catheter comprises a position sensor coupled to the distal tip and configured to produce position signals indicative of the position and orientation of the distal tip in the patient heart. The components of the catheter are described in more detail in FIGS. 1 and 2 below.

In some examples, the processor is configured to receive from the catheter a first plurality of ultrasound (US) images acquired at the intended ablation site of the heart from a first plurality of positions and orientations, before performing the tissue ablation. Note that the plurality of positions and orientations is obtained when a physician moves the catheter relative to the ablation site and uses the catheter to acquire the US images when the distal tip is positioned at the first plurality of distances and orientations relative to the ablation site.

After acquiring the first plurality of US images, the physician uses one or more ablation electrodes (of an ablation catheter, or if available, an ablation electrode of the 4D US catheter) for performing ablation of the tissue at the intended ablation site.

In some examples, after (and optionally, while) performing the ablation, the physician moves the distal tip to revisit the ablation site. In such examples, the processor is configured to receive from the catheter a second plurality of US images acquired at the ablation site, from second of positions and a plurality orientations, (while and) after performing the tissue ablation.

In some examples, the processor is configured to identify among the first and second plurality of US images, one or more pairs of first and second US images, respectively, which are acquired from matched position (i.e., distance) and orientations of the distal tip relative to the ablation site. In other words, the processor identifies for each pair, a pre-ablation (i.e., first) US image, and a post-ablation (i.e., second) US image acquired at least from matched orientation of the distal tip relative to the ablation site. Note that: (i) the term “matched orientation” refers to a difference in at least one of the orientation angles that is smaller than about 10 degrees between the first and second US images, and (ii) a different distance between the first and second identified US images may be compensated by altering the zoom of the image.

In some examples, the processor is configured to select a given pair among the identified pairs, in which the difference between the first and second US images is the largest among the identified pairs. In other words, the processor is configured to select the pair in which the difference between the pre-ablation and the post-ablation US images is the greatest. In some examples, the display is configured to display the given pair to the physician for assessment of the lesion formed in the ablation procedure.

In some examples, the plurality of first (pre ablation) and second (post ablation) US images comprise three-dimensional (3D) US images. In such examples, the processor is configured to apply the image identification and selection technique to one or both of: (i) the 3D US images, and (ii) one or more two-dimensional (2D) slices selected from the 3D US images. In an example, the processor is configured to apply the image identification and selection technique to first and second pairs of pre-ablation and post-ablation US images of first and second 2D slices, respectively, and to display to the physician (over the display device), the first and second given pairs of the respective first and second 2D slices. In other words, the processor may display to the physician at least one given pair from each selected 2D slice (and optionally from the 3D US images) so that the physician can observe the pre- and post-ablation images from different orientations of the respective 2D slices.

The disclosed techniques improve the visualization of lesions formed in tissue, and therefore, improve the quality of ablation procedures.

### System Description

FIG. 1 is a schematic, pictorial illustration of a catheter-based ultrasound imaging and tissue ablation system 10, in accordance with an example of the present disclosure.

In some examples, system 10 may include multiple catheters, which are percutaneously inserted by a physician 24 through the patient's vascular system into a chamber or vascular structure of a heart 12. Typically, a delivery sheath catheter is inserted into a chamber in question, near a desired location in heart 12. Thereafter, one or more catheters can be inserted into the delivery sheath catheter so as to arrive at the desired location within heart 12. The plurality of catheters may include catheters dedicated for sensing Intracardiac Electrogram (IEGM) signals, catheters dedicated for ablating, catheters adapted to carry out both sensing and ablating, and catheters configured to perform imaging of tissues (e.g., tissue 33) of heart 12.

Reference is now made to an inset 17 showing a catheter 14 and a sectional view of heart 12. In some examples, physician 24 may place a distal tip 28 of catheter 14 in close proximity with or contact with the heart wall for performing diagnostics (e.g., imaging and/or sensing) and/or treatment (e.g., tissue ablation) in a target (e.g., ablation) site in heart 12. Additionally, or alternatively, for ablation, physician 24 would similarly place a distal end of an ablation catheter in contact with a target site for ablating tissue intended to be ablated. In the present example shown in inset 17, distal tip 28 is positioned in front of tissue 33 of heart 12.

Reference is now made to an inset 45 showing distal tip 28. In some examples, distal tip 28 comprises a four-dimensional (4D) ultrasound (US) catheter with distal tip 28 having ultrasound transducers 53, which are arranged in a two-dimensional (2D) array 42 and are configured to apply US waves to tissue 33 (and or any other area of heart 12).

In the present example, 2D array 42 comprises about 32×64 US transducers 53 (or any other suitable number of US transducers 53 arranged in any suitable structure), and is configured to produce US-based images of at least tissue 33 located at an inner wall of heart 12.

In some examples, distal tip 28 comprises a position sensor 44 embedded in or near distal tip 28 for tracking position and orientation of distal tip 28 in a coordinate system of system 10. More specifically, position sensor 44 is configured to output position signals indicative of the position and orientation of 2D array 42 inside heart 12. Based on the position signals, a processor 77 of system 10 is configured to display the position and orientation of distal tip 28 over an anatomical map 20 of heart 12, as will be described in more detail below. Optionally and preferably, position sensor 44 comprises a magnetic-based position sensor including three magnetic coils for sensing three-dimensional (3D) position and orientation. The position tracking components of system 10 are described in more detail below.

In some examples, distal tip 28 may be further used to perform the aforementioned diagnostics and/or therapy, such as electrical sensing and/or ablation of tissue 33 in heart 12, using, for example, a tip electrode 46. In the present example, tip electrode 46 may comprise a sensing electrode or an ablation electrode.

In other examples, system 10 may comprise another catheter (not shown) inserted into heart 12 that may have one and preferably multiple electrodes optionally distributed along the distal tip of the respective catheter. The electrodes are configured to sense the IEGM signals and/or electrocardiogram (ECG) signals in tissue 33 of heart 12.

Reference is now made back to the general view of FIG. 1. In some examples, magnetic based position sensor 44 may be operated together with a location pad 25 including a plurality of (e.g., three) magnetic coils 32 configured to generate a plurality of (e.g., three) magnetic fields in a predefined working volume. Real time position of distal tip 28 of catheter 14 may be tracked based on magnetic fields generated with location pad 25 and sensed by magnetic based position sensor 44. Details of the magnetic based position sensing technology are described, for example, in U.S. Pat. Nos. 5,5391,199; 5,443,489; 5,558,091; 6,172,499; 6,239,724; 6,332,089; 6,484,118; 6,618,612; 6,690,963; 6,788,967; 6,892,091.

In some examples, system 10 includes one or more electrode patches 38 positioned for skin contact on patient 23 to establish location reference for location pad 25 as well as impedance-based tracking of electrodes (no shown). For impedance-based tracking, electrical current is directed toward electrode 46, and/or to other electrodes (not shown) of catheter 14, and sensed at electrode skin patches 38 so that the location of each electrode (e.g., electrode 46) can be triangulated via the electrode patches 38. This technique is also referred to herein as Advanced Current Location (ACL) and details of the impedance-based location tracking technology are described in U.S. Pat. Nos. 7,536,218; 7,756,576; 7,848,787; 7,869,865; and 8,456,182.

In some examples, the magnetic based position sensing and the ACL may be applied concurrently, e.g., for improving the position sensing of one or more electrodes coupled to a shaft of a rigid catheter or to flexible arms or splines at the distal tip of another sort of catheter, R such as basket catheter 14, and the PentaRay® or OPTRELL® catheters, available from Biosense Webster, Inc., 31A Technology Drive, Irvine, CA 92618.

In some examples, a recorder 11 displays electrograms 21 captured with body surface ECG electrodes 18 and intracardiac electrograms (IEGM) captured, e.g., with electrode 46 of catheter 14. Recorder 11 may include pacing capability for pacing the heart rhythm and/or may be electrically connected to a standalone pacer.

In some examples, system 10 may include an ablation energy generator 50 that is adapted to conduct ablative energy to one or more of electrodes at a distal tip of a catheter configured for ablating. Energy produced by ablation energy generator 50 may include, but is not limited to, radiofrequency (RF) energy or pulse trains of pulsed-field ablation (PFA) energy, including monopolar or bipolar high-voltage DC pulses as may be used to effect irreversible electroporation (IRE), or combinations thereof. In another example, electrode 46 may comprise an ablation electrode, positioned at distal tip 28 and configured to apply the RF energy and/or the pulse trains of PFA energy to tissue of the wall of heart 12.

In some examples, patient interface unit (PIU) 30 is an interface configured to establish electrical communication between catheters, electrophysiological equipment, power supply and a workstation 55 for controlling the operation of system 10.

Electrophysiological equipment of system 10 may include for example, multiple catheters, location pad 25, body surface ECG electrodes 18, electrode patches 38, ablation energy generator 50, and recorder 11. Optionally and preferably, PIU 30 additionally includes processing capability for implementing real-time computations of location of the catheters and for performing ECG calculations.

In an example, one or more electrodes (e.g., electrode 46) are configured to receive electrical current from PIU 30, and impedance is measured between at least one of the electrodes (e.g., electrode 46) and (i) a respective electrode patch 38, or (ii) a respective body surface ECG electrode 18.

In some examples, workstation 55 includes a storage device, processor 77 with suitable random-access memory, or storage with appropriate operating software stored therein, an interface 56 configured to exchange signals of data (e.g., between processor 77 and another entity of system 10) and user interface capability. In an example, processor 77 is configured to produce a signal indicative of an electrophysiological (EP) property of heart 12. For example, (i) a first a signal indicative of electrical potential measured on the tissue in question having one or more electrodes (not shown) placed in contact therewith, and (ii) a second signal indicative of the measured impedance described above. Workstation 55 may provide multiple functions, optionally including (1) modeling the endocardial anatomy in three-dimensions (3D) and rendering the model or anatomical map 20 for display on a display device 27 (also referred to herein as a display, for brevity), (2) displaying on display device 27 activation sequences (or other data) compiled from recorded electrograms 21 in representative visual indicia or imagery superimposed on the rendered anatomical map 20, (3) displaying real-time location and orientation of multiple catheters within the heart chamber, and (4) displaying on display device 27 anatomical images (e.g., ultrasound images) of sites of interest, such as places where ablation energy has been applied, or intended to be applied.

Reference is now made back to inset 45. In some examples, processor 77 is configured to control distal tip 28 of catheter 14 to: (i) apply ultrasound (US) waves to tissue 33, and (ii) produce signals indicative of the (a) US waves returned from tissue 33, and (b) position signals indicative of the position and orientation of distal tip 28 in the coordinate system of system 10.

One commercial product embodying elements of the system 10 is available as the CARTO™ 3 System, available from Biosense Webster, Inc., 31A Technology Drive, Irvine, CA 92618.

FIG. 2A is a schematic, pictorial illustration of intracardiac ultrasound signals applied to heart tissue 33 before applying ablation energy to an ablation site 63, in accordance with examples of the present disclosure.

In some examples, after performing an electro-anatomical (EA) mapping of at least part of heart 12, processor 77 is configured to display, over map 20, a propagation vector-field indicative of propagation of an electrophysiological (EP) wave over at least tissue 33 of heart 12. Based on the propagation vector-field, processor 77 and/or physician 24 may determine properties (e.g., location, size and orientation) of ablation site 63 intended to be ablated during an ablation procedure. Note that in FIG. 2A, ablation site 63 is shown in a dashed line because ablation energy has not yet been applied to tissue 33 at ablation site 63.

In some examples, before performing the ablation, physician 24 moves distal tip 28 relative to ablation site 63 and applying the US signals for acquiring US images of tissue 33 at least at ablation site 63. In the example of FIG. 2A, 2D array 42 applies the US waves in a three-dimensional (3D) wedge having an azimuthal axis X, an axial axis Y, and an elevation axis Z relative to the apex of the wedge, e.g., a location 58 of 2D array 42. In the context of the present disclosure and in the claims, the terms location and position are used interchangeably, for example, location 58 may be replaced with the term position 58. Moreover, distal tip 28 receives the US waves returned from tissue 33 for producing 3D US images 59 and 60 of tissue 33. In such examples, processor 77 is configured to receive from catheter 14 a first plurality of US images 59 and 60, acquired at the intended ablation site 63 of heart 12. Note that in FIG. 2A the US images are acquired (before performing the tissue ablation) from a first plurality of positions and orientations.

In the example of FIG. 2A, (i) a vector 61 is indicative of the distance and orientation (e.g., angle in the XYZ coordinate axis) of a location 57 of 2D array 42 relative to ablation site 63 for acquiring US image 59, and (ii) a vector 62 is indicative of the distance and orientation (e.g., angle in the XYZ coordinate axis) of location 58 of 2D array 42 relative to ablation site 63 for acquiring US image 60. Note that US images 59 and 60 are shown for the sake of conceptual clarity and physician 24 typically controls system 10 to acquire additional 3D US images at additional positions and orientations relative to ablation site 63.

Note that the XYZ coordinate system is applicable to each 3D US image shown in FIG. 2A, and is FIGS. 2B, 3A, but the apex of the XYZ coordinate system is the position of the 2D array 42 at the respective 3D US image. For example, in image 59 location 57 is the apex of the XYZ coordinate system, and in image 60 location 58 is the apex of the XYZ coordinate system.

FIG. 2B is a schematic, pictorial illustration of intracardiac ultrasound signals applied to heart tissue 33 for assessing the quality of a lesion 66 formed by tissue ablation, in accordance with examples of the present disclosure.

In some examples, after acquiring the first plurality of US images (e.g., images 59 and 60), physician 24 uses one or more ablation electrodes (not shown) of an ablation catheter 65, for producing lesion 66 by applying ablation energy to tissue 33 at the intended ablation site 63 shown in FIG. 2A above. Note that the size, shape, and quality of lesion 66 are determined by an ablation index, which is a novel marker incorporating contact force, time, and power of the ablation in a weighted formula. In the present example, the ablation index is calculated by processor 77 based on several parameters of the ablation process, such as but not limited to: ablation energy, ablation time, and the amplitude and direction of the contact force applied to tissue 33 by catheter 65. An implementation of the ablation index is described in detail, for example, in U.S. Pat. No. 11,304,752, whose disclosure is incorporated herein by reference. In the example of FIG. 2B, a force axis 72 is indicative of the direction of the contact force applied by catheter 65 to tissue 33. In some examples, based on the definition of ablation site 63 (the intended ablation site) processor 77 is configured to determine the direction of force axis 72, so as to obtain the shape of lesion 66, and the ablation index may determine, inter alia, the size of lesion 66.

In some examples, after (and optionally, while) performing the ablation, physician 24 moves distal tip 28 to revisit ablation site 63 for producing additional 3D US images of tissue 33 and lesion 66. In such examples, processor 77 is configured to receive from catheter 14 a second plurality of US images from a second plurality of positions and orientations, (while and) after performing the tissue ablation. In the example of FIG. 2B, 3D US images 69 and 70 are acquired when 2D array 42 is positioned at locations 67 and 68, respectively. Note that typically processor 77 may receive additional 3D US images from additional positions and orientations of distal tip 28 relative to the position of lesion 66, and only two examples thereof (e.g., 3D US images 69 and 70) for illustrating the disclosed techniques.

In accordance with the description in FIG. 2A above, in image 69 location 67 is the apex of the XYZ coordinate system, and in image 70 location 68 is the apex of the XYZ coordinate system.

In the example of FIG. 2B, a vector 71 is indicative of the distance and orientation (e.g., angle in the XYZ coordinate system) between location 67 and lesion 66, and a vector 74 is indicative of the distance and orientation (e.g., angle in the XYZ coordinate system) between location 68 and lesion 66.

In some examples, processor 77 is configured to identify among the first and second plurality of US images (e.g., images 59, 60 and 69, 70 shown in FIGS. 2A and 2B, respectively) one or more pairs of first and second US images, respectively, which are acquired from matched position (i.e., distance) and orientations (e.g., angle) of distal tip 28 relative to ablation site 63 and lesion 66. In other words, processor 77 identifies for each pair, a pre-ablation (i.e., first) US image, and a post-ablation (i.e., second) US image acquired at least from matched orientation of the distal tip relative to ablation site 63 and the position of lesion 66. In the example of FIGS. 2A and 2B, a first pair of images comprises images 59 and 69, and a second pair comprises images 60 and 70.

Note that the term “matched orientation” refers to a difference in at least one of the X, Y and Z axes of the that is smaller than about 10 degrees between the first and second US images. It is noted that when physician 24 moves distal tip 28 relative to ablation site 63 and lesion 66, before and after applying the ablation energy, respectively, the location of 2D array 42 is typically not identical before and after the ablation. Therefore, processor 77 may have a threshold of about 10 degrees, and in case the difference between the directions (in each of the X, Y, and Z angles) of the vectors of pre-ablation and post ablation images is smaller than about 10 degrees, these images have the matched orientation. For example, in case the difference between the orientations of vectors 62 and 74 is smaller than about 10 degrees, images 60 and 70 have a matched orientation. Similarly, the difference between the orientations of vectors 61 and 71 is smaller than about 10 degrees, and therefore, images 59 and 69 have a matched orientation.

Note that a different distance between the position of 2D array 42 in the first and second identified US images may be compensated by altering the zoom of the image. For example, in case the size of vector 62 is larger than that of vector 74 by about 5% or 10%, processor 77 may apply a digital zoom to one or both of images 60 and 70 in order to compensate for the size difference between vectors 62 and 74.

In some examples, processor 77 is configured to select among the 3D US images, at least one pair whose orientation is approximately parallel to force axis 72. In the example of FIG. 2B, vector 74 of image 70 is approximately parallel to force axis 72, whereas the direction of vector 71 is not parallel to force axis 72. The inventors found that the quality of an image, whose orientation is approximately parallel to the force axis of the catheter performing tissue ablation, is typically improved relative to that of an image whose orientation is not parallel to the force axis. In the example of FIG. 2B, processor 77 is configured to select the pair of images 60 and 70, over the pair of images 59 and 69. FIGS. 3A and 3B below describe additional techniques for obtaining two-dimensional (2D) images of ablation site 63 and lesion 66 based on images 60 and 70, respectively, however, processor 77 may apply the same techniques, mutatis mutandis, to other pairs of 3D US images, such as images 59 and 69.

In some examples, processor 77 is configured to compare between images 59 and 69, and between images 60 and 70 in order to assess the quality of lesion 66. More specifically, processor 77 is configured to select among the two pairs, one pair in which the difference between the pre-ablation and post ablation US images is the largest. For example, processor 77 may use a structural similarity index (SSIM), and/or analysis of the grayscale histogram (intensity histogram) of the respective images in order to select the pair in which the difference between the pre-ablation and post ablation US images is the largest. In other examples, processor 77 may use any other suitable type of on volumetric analysis algorithmic techniques for comparing between the pairs of pre-ablation and post-ablation US images, such as between images 59 and 69, and between images 60 and 70.

In the example of FIGS. 2A and 2B, processor 77 selects the pair of images 60 and 70 in which the difference therebetween is the largest among all other pairs of 3D US images, such as images 59 and 69. It is noted that the difference between images 60 and 70 may provide physician 24 with sufficient information for assessing the effect of the ablation energy on tissue 33, and more specifically, on the quality of lesion 66.

In some examples, display device 27 is configured to display to physician 24, 3D US images 60 and 70, which are selected by processor 77 for having the largest difference between a pre-ablation and post-ablation 3D US images. In other examples, processor 77 may select 2D images of ablation site 63 and lesion 66, which are based on images 60 and 70, as described in detail in FIGS. 3A and 3B below.

FIGS. 3A and 3B are schematic, pictorial illustrations of images 60 and 70 of heart tissue 33 analyzed for assessing the quality of lesion 66, in accordance with examples of the present disclosure. FIG. 3A comprises 3D US image 60 acquired before the ablation of tissue 33 at ablation site 63, and FIG. 3B comprises 3D US image 70 acquired after the tissue ablation and the formation of lesion 66 at ablation site 63.

As described in FIGS. 2A and 2B above, processor 77 compared between 3D US images 59 and 69, and between 3D US images 60 and 70, but the 3D US images may not provide physician 24 with sufficient information for assessing the quality of lesion 66.

In some examples, processor 77 is configured to select one or more pairs of 2D slices of the 3D US images 60 and 70, each pair of 2D slices comprises 2D US images of (i) ablation site 63 and (ii) lesion 66, which are obtained from 3D US images 60 and 70, respectively. In the example of FIG. 3A, processor 77 selects 2D slices 73 and 78, and in the example of FIG. 3B, processor 77 selects 2D slices 83 and 88 corresponding to 2D slices 73 and 78, respectively. Note that the 2D US images of slices 73 and 78 comprise at least ablation site 63 and the surrounding thereof, and the 2D US images of slices 83 and 88 comprise at least lesion 66 and the surrounding thereof. More specifically, (i) 2D slices 73 and 83 comprise a first pair of 2D US images derived from 3D US images 60 and 70, which are acquired before and after the ablation, respectively, and (ii) 2D slices 78 and 88 comprise a second pair of 2D US images that are also derived from 3D US images 60 and 70 that have been acquired before and after the ablation, respectively.

In some examples, processor 77 is configured to apply the image identification and selection technique described in FIG. 2B above, mutatis mutandis, to the first and second pairs of pre-ablation and post-ablation US images of first and second 2D slices, respectively. More specifically, processor 77 is configured to apply the SSIM and/or the grayscale histogram analysis (described in FIG. 2B above) to check the difference: (i) between the 2D US images of slices 73 and 83, and (ii) between the 2D US images of slices 78 and 88.

In some examples, based on the comparison, processor 77 is configured to select a pair of the 2D US images having the largest difference between the 2D images thereof. In the example of FIGS. 3A and 3B, processor 77 selects the 2D US images of slices 78 and 88.

Reference is now made back to FIGS. 2A and 2B. In some examples, the comparison between the 3D US images (e.g., between images 60 and 70) as described in FIG. 2B above, may also rely on information obtained from one or more 2D slices derived from the volumetric US images (e.g., from images 60 and 70) of tissue 33 at the ablation site. For example, processor 77 may apply the SSIM and/or grayscale histogram analysis to the 2D US image of slices 73, 78, 83 and 88 (and optionally additional 2D slides derived from images 60 and 70), in conjunction with volumetric-based analysis techniques, for comparing between images 59 and 69, and between images 60 and 70.

FIG. 4 is a schematic, pictorial illustration of 2D US images 81 and 82 acquired before and after tissue ablation and displayed over display device 27, in accordance with an example of the present disclosure.

As described in the example of FIGS. 3A and 3B above, processor 77 selects the 2D slices 78 and 88 whose pair of 2D US images have the largest difference among the other pairs of slices (e.g., slices 73 and 83).

In some examples, display device 27 is configured to display (e.g., to physician 24) 2D US images 81 and 82 of 2D slices 78 and 88, respectively. In the example of FIG. 4, images 81 and 82 are displayed side-by-side, but in other examples, processor 77 and/or display device 27 may use any other suitable arrangement of images 81 and 82.

In some examples, processor 77 is configured to present over images 81 and 82 markers indicative of a measurement of the thickness of tissue 33 before and after ablation, as shown for example in regions 87 and 84 of images 81 and 82, respectively. Processor 77 is further configured to present, e.g., over image 82 (post ablation image), ablation index 86 and a tag 85 indicative of the ablation index, or any other suitable type of tag.

In other examples, processor 77 is configured to select more than a single pair of pre-ablation and post-ablation US images. For example, display device 27 is configured to display 2D US images 81 and 82 together with 3D US images 60 and 70, which are both selected by processor 77. Additionally, or alternatively, processor 77 may select two or more pairs of 2D slices (e.g., slices 73 and 83, and slices 78 and 88), and display device 27 may display the 2D US images thereof in two pairs, so that physician 24 may perform the assessment based on two or more pairs of images acquired before and after the ablation. In other words, processor 77 may display to physician 24 at least a pair of 2D US images from each selected 2D slice (and optionally from the 3D US images) so that physician 24 can observe the pre- and post-ablation images from different orientations of the respective 2D slices (and optionally, also the volumetric US images).

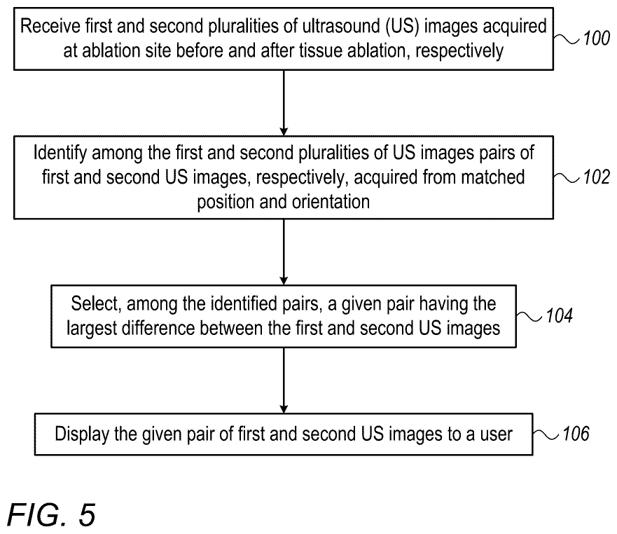

FIG. 5 is a flow chart that schematically illustrates a method for assessing lesion 66 using one or more pairs of selected US images, in accordance with an example of the present disclosure.

The method begins at an ultrasound image acquisition step 100 with: (i) the insertion and movement of distal tip 28 into heart 12 (e.g., by physician 24) for acquiring a first plurality of 3D US images (e.g., 3D US images 59 and 60) of tissue 33 at ablation site 63, (ii) performing tissue ablation and forming lesion 66 at the location of ablation site 63, and (iii) acquiring a second plurality of 3D US images (e.g., 3D US images 69 and 70) of tissue 33 and lesion 66. In some examples, processor 77 receives from catheter 14 the first and second pluralities of US images acquired at ablation site 63 before and after the tissue ablation, respectively. The sequence of step 100 described in detail in FIGS. 2A and 2B above.

At an image identification step 102, processor 77 identifies, among the first and second pluralities of US images, one or more pairs of first and second US images, respectively, which are acquired from matched position and orientation. For example, processor 77 identifies 3D US images 60 and 70 acquired from matched position and orientations. More specifically, when acquiring images 60 and 70, the respective positions and orientations of 2D array 42 at locations 58 and 68, relative to ablation site 63 and lesion 66, respectively, are defined by vectors 62 and 74 having similar size and direction. The same technique applies to vectors 61 and 71 of images 59 and 69, respectively, as shown, and also described in FIGS. 2A and 2B above. Moreover, processor 77 is configured to derive one or more 2D slices of US images from the 3D US images, and define or identify pairs of 2D US images, such as the 2D US images of: (i) slices 73 and 83, and (ii) slices 78 and 88, as shown and described in detail in FIGS. 3A and 3B above.

At a pair selection step 104, processor selects, among the identified pairs, a given pair having the largest difference between the first and second US images described in step 102 above. Note that the comparison may be perform on pairs of 3D US images as well as on pairs of 2D US images, as described in detail in FIGS. 2A, 2B, 3A and 3D. More specifically, (i) the difference between 3D US images 60 and 70, is larger than the difference between 3D US images 59 and 69, and (ii) the difference between 2D the US images of slices 78 and 88 (i.e., images 81 and 82 of FIG. 4, respectively), is larger than the difference between 2D US images of slices 73 and 83.

At a displaying step 106 that terminates the method, display device 27 displays the selected pairs of 2D US images and/or 3D US images to physician 24 and other optional users of system 10, as described in detail in FIG. 4 above. Moreover, processor 77 and display device 27 are further configured to present ablation tags (e.g., ablation tag 85), the calculated value (s) of ablation index 86, and optionally, additional information over the selected 2D and/or 3D US images.

Although the examples described herein mainly address visualization and assessment of lesions formed in tissue ablation procedures carried out in patient heart, the methods and systems described herein can also be used in other applications, such as in visualization and assessment of lesions formed in tissue ablation procedures carried out in organs other than the heart. Moreover, the disclosed techniques may be used for visualizing and assessing the outcome of any medical (e.g., surgical) procedures associated with altering at least one of the size, shape, and morphology of any suitable organ of a patient.

### Example 1

A system (10), comprising:

a processor (77), which is configured to:
receive a first plurality of ultrasound (US) images (59, 60) acquired at a site (63) of an organ (12) from a first plurality of positions (57, 58) and orientations (61, 62), before performing a medical procedure at the site;
receive a second plurality of US images (69, 70) acquired at the site, from a second plurality of positions (67, 68) and orientations (71, 74), after performing the medical procedure at the site; and
identify among the first and second plurality of US images, one or more pairs of first and second US images, respectively, which are acquired from matched orientations (62, 74), and select a given pair among the pairs, in which a difference between the first and second US images (81, 82) is largest among the identified pairs; and
a display (27), which is configured to display the given pair of the first and second US images to a user (24).

### Example 2

The system according to Example 1, and comprising a catheter, which is configured to be inserted into the organ and to acquire the first and second plurality of US images, and wherein the processor is configured to identify, among the one or more pairs, at least one pair of the first and second US images acquired from a matched position of a distal tip of the catheter.

### Example 3

The system according to Example 2, wherein the catheter comprises: (i) a two-dimensional (2D) ultrasound transducer array, and (ii) a position sensor configured to output position signals indicative of a position and an orientation of the 2D ultrasound transducer array inside the organ, and wherein the processor is configured to identify the one or more pairs having the matched orientations and the matched positions based on the position signals received from the position sensor.

### Example 4

The system according to any of Examples 1 and 2, wherein the first and second pluralities of US images are produced based on first and second pluralities of three-dimensional (3D) US images, each of the 3D US images having first, second and third orientations, corresponding to three dimensions of the 3D US images, and wherein the processor is configured to identify the one or more pairs acquired from matched orientation by comparing between the first, second and third orientations of the one or more pairs of first and second US images.

### Example 5

The system according to Example 4, wherein the processor is configured to: (i) identify the one or more pairs by selecting in the first and second pluralities of 3D US images, one or more pairs of first and second two-dimensional (2D) US images, respectively, of a 2D slice having the matched first, second and third orientations, (ii) calculate the difference between the first and second 2D US images of each pair of the 2D slice, and (iii) select the given pair having the largest difference.

### Example 6

The system according to Example 5, wherein the processor is configured to: (i) select an additional set of additional one or more pairs of first and second 2D US images of an additional 2D slice having another matched first, second and third orientations, (ii) calculate the difference between the additional first and second 2D US images of each additional pair of the 2D slice, and (iii) select an additional given pair having the largest difference, and wherein the display is configured to display at least one of the given pair and the additional given pair to the user.

### Example 7

The system according to any of Examples 1 and 2, wherein the first and second US images comprise pairs of first and second three-dimensional (3D) US images, respectively, which are acquired from matched orientations, and wherein the processor is configured to select among the pairs, the given pair of the first and second 3D US images in which the difference between the first and second 3D US images is largest among the identified pairs.

### Example 8

The system according to any of Examples 1 and 2, wherein the display is configured to display over the first and second US images of the given pair, at least a mark indicative of an attribute of the organ at the site.

### Example 9

The system according to Example 8, wherein the organ comprises a heart and the medical procedure comprises tissue ablation at the site in the heart, and wherein the mark comprises one or both of: (i) an ablation tag indicative of a lesion formed at the site, and (ii) an ablation index indicative of parameters of the ablation.

### Example 10

The system according to any of Examples 1 and 2, wherein the processor is configured to estimate the difference between the first and second US images of the identified pairs using at least one image analysis tool selected from: (i) a structural similarity index (SSIM), and (ii) analysis of a grayscale intensity histogram.

### Example 11

A method, comprising:
receiving a first plurality of ultrasound (US) images (59, 60) acquired at a site (63) of an organ (12) from a first plurality of positions (57, 58) and orientations (61, 62), before performing a medical procedure at the site;
receiving a second plurality of US images (69, 70) acquired at the site, from a second plurality of positions (67, 68) and orientations (71, 74), after performing the medical procedure at the site; and
identifying among the first and second plurality of US images, one or more pairs of first and second US images, respectively, which are acquired from matched orientations (62, 74), and selecting a given pair among the pairs, in which a difference between the first and second US images (81, 82) is largest among the identified pairs; and
displaying the given pair of the first and second US images to a user (24).

It will be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A system, comprising:
a processor, which is configured to:
receive a first plurality of ultrasound (US) images acquired at a site of an organ from a first plurality of positions and orientations, before performing a medical procedure at the site;
receive a second plurality of US images acquired at the site, from a second plurality of positions and orientations, after performing the medical procedure at the site; and
identify among the first and second plurality of US images, one or more pairs of first and second US images, respectively, which are acquired from matched orientations, and select a given pair among the pairs, in which a difference between the first and second US images is largest among the identified pairs; and
a display, which is configured to display the given pair of the first and second US images to a user.

2. The system according to claim 1, and comprising a catheter, which is configured to be inserted into the organ and to acquire the first and second plurality of US images, and wherein the processor is configured to identify, among the one or more pairs, at least one pair of the first and second US images acquired from a matched position of a distal tip of the catheter.

3. The system according to claim 2, wherein the catheter comprises: (i) a two-dimensional (2D) ultrasound transducer array, and (ii) a position sensor configured to output position signals indicative of a position and an orientation of the 2D ultrasound transducer array inside the organ, and wherein the processor is configured to identify the one or more pairs having the matched orientations and the matched positions based on the position signals received from the position sensor.

4. The system according to claim 1, wherein the first and second pluralities of US images are produced based on first and second pluralities of three-dimensional (3D) US images, each of the 3D US images having first, second and third orientations, corresponding to three dimensions of the 3D US images, and wherein the processor is configured to identify the one or more pairs acquired from matched orientation by comparing between the first, second and third orientations of the one or more pairs of first and second US images.

5. The system according to claim 4, wherein the processor is configured to: (i) identify the one or more pairs by selecting in the first and second pluralities of 3D US images, one or more pairs of first and second two-dimensional (2D) US images, respectively, of a 2D slice having the matched first, second and third orientations, (ii) calculate the difference between the first and second 2D US images of each pair of the 2D slice, and (iii) select the given pair having the largest difference.

6. The system according to claim 5, wherein the processor is configured to: (i) select an additional set of additional one or more pairs of first and second 2D US images of an additional 2D slice having another matched first, second and third orientations, (ii) calculate the difference between the additional first and second 2D US images of each additional pair of the 2D slice, and (iii) select an additional given pair having the largest difference, and wherein the display is configured to display at least one of the given pair and the additional given pair to the user.

7. The system according to claim 1, wherein the first and second US images comprise pairs of first and second three-dimensional (3D) US images, respectively, which are acquired from matched orientations, and wherein the processor is configured to select among the pairs, the given pair of the first and second 3D US images in which the difference between the first and second 3D US images is largest among the identified pairs.

8. The system according to claim 1, wherein the display is configured to display over the first and second US images of the given pair, at least a mark indicative of an attribute of the organ at the site.

9. The system according to claim 8, wherein the organ comprises a heart and the medical procedure comprises tissue ablation at the site in the heart, and wherein the mark comprises one or both of: (i) an ablation tag indicative of a lesion formed at the site, and (ii) an ablation index indicative of parameters of the ablation.

10. The system according to claim 1, wherein the processor is configured to estimate the difference between the first and second US images of the identified pairs using at least one image analysis tool selected from: (i) a structural similarity index (SSIM), and (ii) analysis of a grayscale intensity histogram.

11. A method, comprising:
receiving a first plurality of ultrasound (US) images acquired at a site of an organ from a first plurality of positions and orientations, before performing a medical procedure at the site;
receiving a second plurality of US images acquired at the site, from a second plurality of positions and orientations, after performing the medical procedure at the site; and
identifying among the first and second plurality of US images, one or more pairs of first and second US images, respectively, which are acquired from matched orientations, and selecting a given pair among the pairs, in which a difference between the first and second US images is largest among the identified pairs; and
displaying the given pair of the first and second US images to a user.

12. The method according to claim 11, and comprising inserting a catheter into the organ and acquiring the first and second plurality of US images, and comprising identifying, among the one or more pairs, at least one pair of the first and second US images acquired from a matched position of a distal tip of the catheter.

13. The method according to claim 12, wherein the catheter comprises: (i) a two-dimensional (2D) ultrasound transducer array, and (ii) a position sensor for outputting position signals indicative of a position and an orientation of the 2D ultrasound transducer array inside the organ, and identifying the one or more pairs having the matched orientations and the matched positions based on the position signals received from the position sensor.

14. The method according to claim 11, and comprising producing the first and second pluralities of US images based on first and second pluralities of three-dimensional (3D) US images, each of the 3D US images having first, second and third orientations, corresponding to three dimensions of the 3D US images, and wherein identifying the one or more pairs acquired from matched orientation comprises comparing between the first, second and third orientations of the one or more pairs of first and second US images.

15. The method according to claim 14, wherein identifying the one or more pairs comprises selecting in the first and second pluralities of 3D US images, one or more pairs of first and second two-dimensional (2D) US images, respectively, of a 2D slice having the matched first, second and third orientations, and wherein selecting the given pair having the largest difference comprises calculating the difference between the first and second 2D US images of each pair of the 2D slice.

16. The method according to claim 15, and comprising: (i) selecting an additional set of additional one or more pairs of first and second 2D US images of an additional 2D slice having another matched first, second and third orientations, (ii) calculating the difference between the additional first and second 2D US images of each additional pair of the additional 2D slice, selecting an additional given pair having the largest difference, and (iv) displaying at least one of the given pair and the additional given pair to the user.

17. The method according to claim 11, wherein identifying the first and second US images comprises identifying pairs of first and second three-dimensional (3D) US images, respectively, which are acquired from matched orientations, and selecting among the pairs, the given pair of the first and second 3D US images in which the difference between the first and second 3D US images is largest among the identified pairs.

18. The method according to claim 11, and comprising displaying over the first and second US images of the given pair, at least a mark indicative of an attribute of the organ at the site.

19. The method according to claim 18, wherein the organ comprises a heart and the medical procedure comprises tissue ablation at the site in the heart, and wherein displaying the mark comprises displaying one or both of: (i) an ablation tag indicative of a lesion formed at the site, and (ii) an ablation index indicative of parameters of the ablation.

20. The method according to claim 11, and comprising using at least one image analysis tool selected from: (i) a structural similarity index (SSIM), and (ii) analysis of a grayscale intensity histogram, for estimating the difference between the first and second US images of the identified pairs.
